(19) United States
(12) Patent Application Publication
(10) Pub. No.: US 2016/0100847 A1
(43) Pub. Date: Apr. 14, 2016

(54) Patello-Femoral With Reusable Instruments

(71) Applicant: Biomet Sports Medicine, LLC, Warsaw, IN (US)

(72) Inventor: William MAXSON, Fort Wayne, IN (US)

(21) Appl. No.: 14/512,960

(22) Filed: Oct. 13, 2014

(51) Int. Cl. A61B 17/17; A61B 17/15; A61B 17/16

(52) U.S. Cl. CPC A61B 17/1714; A61B 17/1615; A61B 17/155; A61B 17/1677; A61B 17/1675; A61B 17/1767; A61B 17/1764

(57) ABSTRACT

A system for repairing a cartilage defect. The system includes a riser block having a bone-engaging side that is a patient-specific negative of a patient's bone anatomy proximate to the cartilage defect. A drill guide-engaging side of the riser block is opposite to the bone-engaging side. A superior riser block guide hole is defined by the riser block and extends from the drill guide-engaging side to the bone-engaging side. An inferior riser block guide hole is defined by the riser block and extends from the drill guide-engaging side to the bone-engaging side.

### FIELD

The present disclosure relates to methods and devices for implanting an implant in bone to repair a cartilage defect.

### BACKGROUND

This section provides background information related to the present disclosure, which is not necessarily prior art.

Articular cartilage enables bones to move smoothly relative to one another, such as in a knee joint. Articular cartilage can be damaged in a variety of different ways, such as by injury (e.g., tearing), by excessive wear, or by a lifetime of use. Damage to the articular cartilage can also cause damage to the underlying bone. In some instances, the damaged articular cartilage can result in pain and reduced mobility. Various surgical procedures have been developed to repair damaged articular cartilage, such as microfracture, mosaicplasty, or a unicondylar or partial knee replacement. While these surgical procedures are effective for their intended purpose, they are subject to improvement.

For example, some systems require a femoral implant to be positioned so that it is flush with surrounding cartilage. This can require either measuring or locating a guide to a best fit position by eye-balling the guide on the femur. The guide is then pinned to the femur and the femur is reamed. There is some variability in this procedure and it may not fully determine which implant and location provides the best fit to position the implant flush with the cartilage. Such a procedure is therefore subject to improvement.

### SUMMARY

This section provides a general summary of the disclosure, and is not a comprehensive disclosure of its full scope or all of its features.

The present teachings provide for a system for repairing a cartilage defect. The system includes a riser block having a bone-engaging side that is a patient-specific negative of a patient's bone anatomy proximate to the cartilage defect. A drill guide-engaging side of the riser block is opposite to the bone-engaging side. A superior riser block guide hole is defined by the riser block and extends from the drill guide-engaging side to the bone-engaging side. An inferior riser block guide hole is defined by the riser block and extends from the drill guide-engaging side to the bone-engaging side.

The present teachings further provide for a system for repairing a cartilage defect. The system includes a riser block having the following: a patient-specific bone-engaging side; a drill guide-engaging side that is opposite to the patient-specific bone-engaging side; a superior riser block guide hole; and an inferior riser block guide hole. Both the superior and inferior riser block guide holes are defined by and extend through the riser block from the drill guide-engaging side to the patient-specific bone-engaging side. The system further includes a drill guide having the following: an outer side; a riser-engaging side opposite to the outer side, the riser-engaging side is complementary to the drill guide-engaging side of the riser block; a superior drill guide hole; and an inferior drill guide hole, both of the superior and inferior drill guide holes extend through the riser block and are arranged to align with the superior and inferior riser block guide holes, respectively, when the drill guide is coupled to the riser block. A reamer is configured to be guided to the bone surface by the superior and inferior drill guide holes, and the superior and inferior riser block guide holes, to prepare the bone to receive an implant.

The present teachings also include a method for repairing a cartilage defect. The method includes the following: imaging a bone of a patient proximate to the cartilage defect; positioning a riser block on the bone proximate to the cartilage defect such that a patient-specific bone-engaging side of the riser block based on the imaging mates with the bone; positioning a drill guide such that a riser-engaging side of the drill guide abuts a drill guide-engaging side of the riser block, and such that superior and inferior drill guide holes defined by the drill guide are respectively aligned with superior and inferior riser block guide holes of the riser block; coupling the riser block and the drill guide together and to the bone with coupling members extending through the drill guide, through the riser block, and into the bone; inserting a reamer through the superior drill guide hole and the superior riser block guide hole to guide the reamer to a superior portion of the bone, and reaming the superior portion of the bone to receive a portion of an implant; inserting the reamer through the inferior drill guide hole and the inferior riser block guide hole to guide the reamer to a superior portion of the bone, and reaming the superior portion of the bone to receive a portion of the implant; and implanting the implant in the bone such that an articular surface of the implant is generally flush with surrounding cartilage.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 illustrates a bone with a cartilage defect, particularly a femur with a defect at a trochlear groove thereof;

FIG. 2 is an imaged model of a femur with a cartilage defect at a trochlear groove thereof, such as the femur of FIG. 1, and includes a model of a femoral implant positioned within the defect;

FIG. 3A is a perspective view of a patient-specific riser block;

FIG. 3B is another perspective view of the patient-specific riser block of FIG. 3A;

FIG. 4 is a perspective view of a drill guide seated on the patient-specific riser block of FIGS. 3A and 3B, with the patient-specific riser block seated on the bone of FIG. 1 over the cartilage defect;

FIG. 5 is a perspective view of the patient-specific riser block and the drill guide secured to the femur with guide pins, and a reamer guided to the bone by the drill guide; and

FIG. 6 illustrates an exemplary implant configured to be implanted in the femur at the cartilage defect.

Corresponding reference numerals indicate corresponding parts throughout the several views of the drawings.

### DETAILED DESCRIPTION

Example embodiments will now be described more fully with reference to the accompanying drawings.

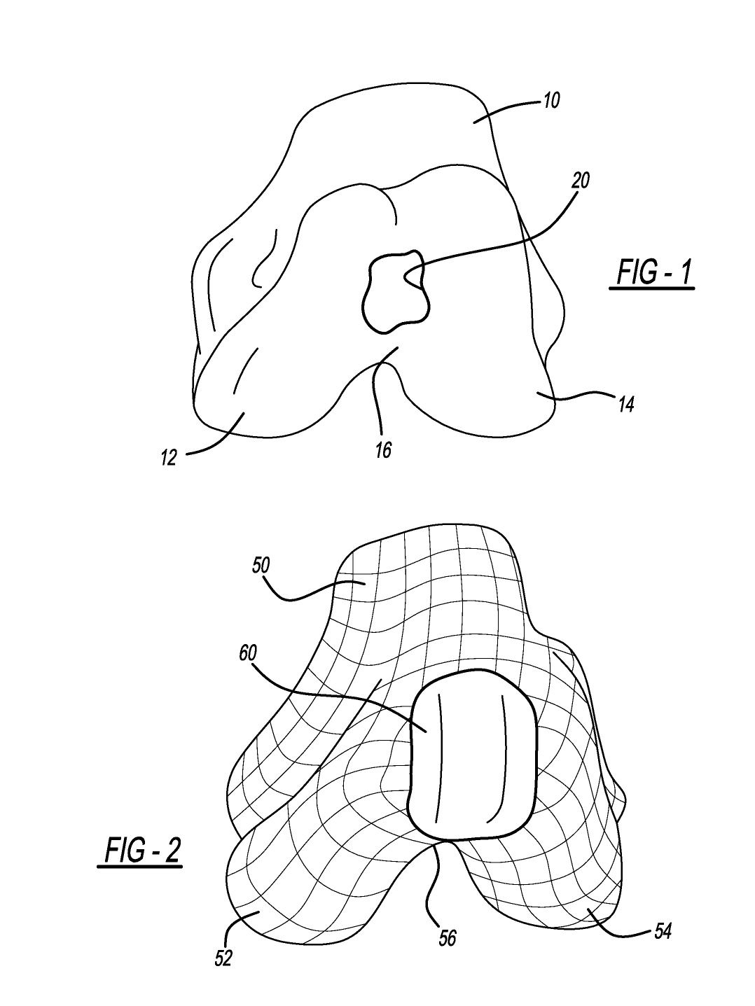

With initial reference to FIG. 1, an exemplary bone, illustrated in this example as a femur, is designated at reference numeral 10. An distal portion of the femur 10 is illustrated, which includes a first condyle 12, a second condyle 14, and a trochlear groove 16 therebetween. Cartilage proximate to the trochlear groove 16 has a defect at reference numeral 20. The defect 20 can be any cartilage defect, such as a tear. The present teachings provide for devices, systems, and methods for repairing the cartilage defect 20. Although the cartilage defect 20 is described herein as associated with a femur 10, the present teachings are applicable for repair of any suitable cartilage defect proximate to any suitable bone surface. For example, cartilage defects at a hip joint or shoulder joint may also be repaired using the present teachings.

To facilitate selection of an implant to be implanted into the femur 10 at or proximate to the cartilage defect 20, a patient's femur 10 is imaged using any suitable imaging technique, such as by using MRI imaging, CT scan imaging, or x-ray imaging, for example. The femur 10 can then be displayed as a femur image 50 on any suitable display using any suitable software. The femur image 50 is an exact replica of the patient's femur 10 and surrounding cartilage, and thus includes an imaged first condyle 52, an imaged second condyle 54, and an imaged trochlear groove 56.

Using the imaging software, an implant model 60 can be selected from a plurality of implants of different shapes and sizes. The implant model 60, as well as any of the other imaged implants, can be selected and virtually positioned on the femur image 50 at the area of the cartilage defect 20 in order to select the implant model 60 having the best fit for the femur 10 and the cartilage defect 20. The implant model 60 can be selected based on a variety of parameters, such as size and shape. In particular, the implant model 60 will be selected such that an articular surface thereof will be generally flush with an outer cartilage surface surrounding the cartilage defect 20.

In the example illustrated, the selected implant model 60 corresponds to a trochlear groove femoral implant, such as the femoral implant 70 illustrated in FIG. 6. The femoral implant 70 generally includes a bone-engaging side 72 and an articular side 74, which is opposite to the bone-engaging side. The articular side 74 includes an articular surface 76, which is configured to articulate with a patella bone. The bone-engaging side 72 includes a projection 78, which is configured to be implanted into the femur 10 at or proximate to the cartilage defect 20. The femoral implant 70 of FIG. 6 is provided for exemplary purposes only, and thus any suitable implant can be implanted in the femur 10 to repair the cartilage defect 20, such as on the condyles, trochlear groove, etc.

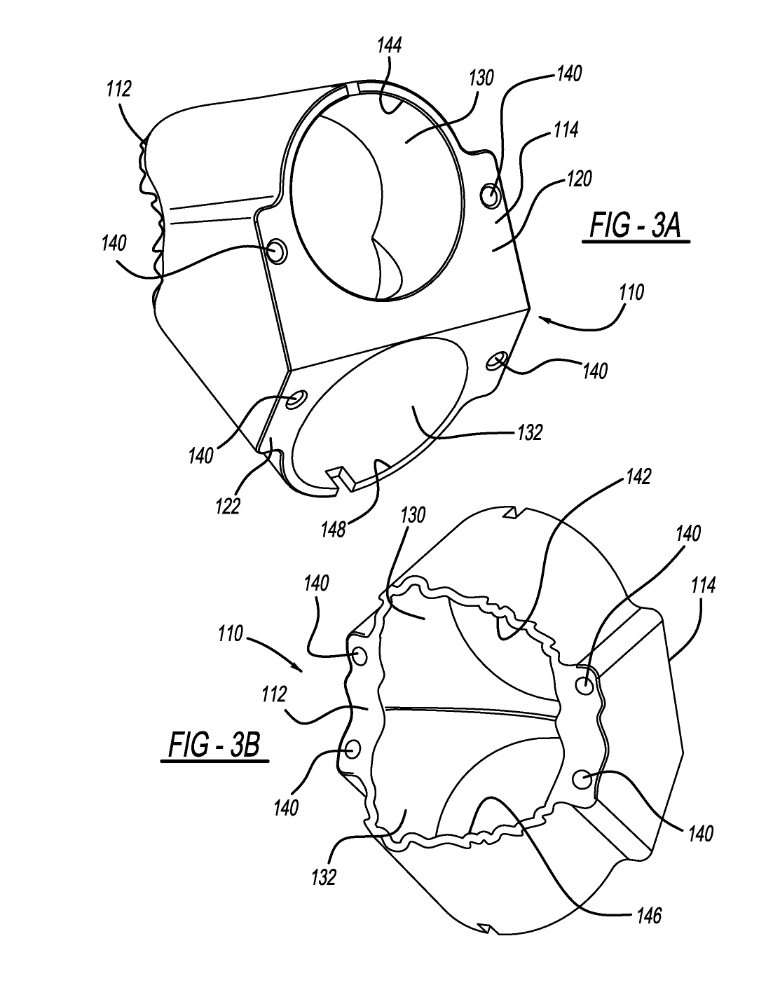

With additional reference to FIGS. 3A, 3B, 4, and 5, the present teachings further include a patient-specific riser block 110. The riser block 110 generally includes a first or bone-engaging side 112 and a second or drill guide-engaging side 114, which is opposite to the bone-engaging side 112. The bone-engaging side 112 is patient-specific, and thus is a negative of the patient's femur 10 that is sized, shaped, and configured to nest in only one position proximate to the cartilage defect 20, such as at the trochlear groove 16.

The patient-specific bone-engaging side 112 is based on two-dimensional image data of the specific patient, such as x-ray data. The x-ray data can include any suitable number of x-ray images or scans, such as only two or more. The x-rays can be taken along first and second intersecting planes, or at any other suitable orientation or position. The patient-specific bone-engaging side 112 can be based directly on the image data (such as x-ray image data) or based on a three-dimensional model, which is based on the two-dimensional image data. The patient-specific bone-engaging side 112 can also include patient-generic portions, which are not based on patient image data, or are based on patient image data having fewer details than the image data used for the patient-specific portions.

The patient-generic portions at the bone-engaging side 112 can be at least partially modeled based on historical data collected from preparation of previous patient-specific guides. For example, bone dimensions collected from patients of a similar sex, height, weight, age, and race can be used to estimate bone dimensions of a new patient of a similar body type and background. The patient-generic portions can be modeled to best-fit the patient based on the available data. A three-dimensional bone model can be made based on the historical data, and the patient-generic portions can then be made based on the three-dimensional model. Alternatively, the patient-generic portions can be made based directly on the historical data.

The patient-specific portions of the patient-specific bone-engaging side 112 of the alignment guides are designed and prepared preoperatively using anatomic landmarks, such as osteophytes, for example, and can be mounted intra-operatively without any registration or other guidance based on their unique patient-specific surface guided by the patient's anatomic landmarks. In other words, the bone-engaging side 112 is patient-specific, and thus is a negative of the patient's femur 10 that is sized, shaped, and configured to nest in only one position at the superior end of the femur 10, such as proximate to the cartilage defect 20 at the trochlear groove 16, for example.

The patient-specific riser block 110 can also include resection or cutting formations in addition to those described herein, such as cutting slots or cutting edges or planes used for guiding a cutting blade to perform bone resections directly through the riser block 110. The riser block 110 can be used in minimally invasive surgery. Various alignment/resection guides and preoperative planning procedures are disclosed in commonly assigned U.S. Pat. No. 8,092,465, filed on May 31, 2007; U.S. Pat. No. 8,608,748, filed Sep. 16, 2008; U.S. Pat. No. 8,070,752, filed on Jan. 9, 2008, U.S. Pat. No. 7,780,672, filed on Feb. 27, 2006; and U.S. Pat. No. 8,298,237, filed Feb. 4, 2008. The disclosures of the above applications are incorporated herein by reference.

As disclosed, for example, in the above-referenced U.S. Pat. No. 8,092,465, filed on May 31, 2007, in the preoperative planning stage, a series of x-rays of the relevant anatomy of the patient can be performed at a medical facility or doctor's office. The scan data obtained can be sent to a manufacturer. The scan data can be used to construct a three-dimensional image of the joint and provide an initial implant fitting and alignment in a computer file form or other computer representation. The initial implant fitting and alignment can be obtained using an alignment method, such as alignment protocols used by individual surgeons.

The outcome of the initial fitting is an initial surgical plan that can be printed or provided in electronic form with corresponding viewing software. The initial surgical plan can be surgeon-specific, when using surgeon-specific alignment protocols. The initial surgical plan, in a computer/digital file form associated with interactive software, can be sent to the surgeon, or other medical practitioner, for review. The surgeon can incrementally manipulate the position of images of various implant components, such as the implant model 60, in an interactive image of the joint. Additionally, the surgeon can select or modify resection planes, types of implants and orientations of implant insertion. After the surgeon modifies and/or approves the surgical plan, the surgeon can send the final, approved plan to the manufacturer.

After the surgical plan is approved by the surgeon, bone preparation guides, such as the riser block 110, including patient-specific areas, such as patient-specific bone-engaging side 112, can be designed by configuring and using a CAD program or other imaging software, according to the surgical plan. Computer instructions of tool paths for machining the patient-specific alignment guides can be generated and stored in a tool path data file.

The tool path can be provided as input to a CNC mill or other automated machining system, and the alignment guides, such as the riser block 110, can be machined from polymer, ceramic, metal or other suitable material. The guides can also be manufactured by various other methods, stereolithography, laser deposition, printing, and rapid prototyping methods. The alignment guides are sterilized and shipped to the surgeon or medical facility for use during the surgical procedure. Various patient-specific knee alignment guides and associated methods are disclosed in the commonly assigned U.S. application Ser. No. 11/756,057, filed on May 31, 2007 (issued as U.S. Pat. No. 8,092,465 on Jan. 10, 2012), which is incorporated herein by reference.

The riser block 110 includes, at the drill guide-engaging side 114, a superior surface 120 and an inferior surface 122. The superior surface 120 and the inferior surface 122 extend away from one another at an angle such that the superior and inferior surfaces 120 and 122 are furthest from the bone-engaging side 112 generally where the superior surface 120 abuts the inferior surface 122. The superior and inferior surfaces 120 and 122 are generally closest to the bone-engaging side 112 at ends thereof that are opposite to ends of the superior and inferior surfaces 120 and 122 that abut one another.

The patient-specific riser block 110 further includes, and defines, a superior riser block guide hole 130 and an inferior riser block guide hole 132. Both the superior and inferior riser block guide holes 130 and 132 extend through the riser block 110 between the bone-engaging side 112 and the drill guide-engaging size 114. Specifically, the bone-engaging side 112 defines a first opening 142 of the superior riser block guide hole 130, and the drill guide-engaging side 114 defines a second opening 144 of the superior riser block guide hole 130. Similarly, the bone-engaging side 112 defines a first opening 146 of the inferior riser block guide hole 132, and the inferior surface 122 of the drill guide-engaging side 114 defines a second opening 148 of the inferior riser block guide hole 132. The superior and inferior riser block guide holes 130 and 132 are angled such that they are furthest apart at the drill guide-engaging side 114, and closest together at the bone-engaging side 112 where they intersect one another.

The patient-specific riser block 110 further includes a plurality of guide bores 140, which extend between the bone-engaging side 112 and the drill guide-engaging side 114. The guide bores 140 are configured to receive suitable coupling members therethrough, such as guide pins or K-wires 190 described herein, in order to secure the riser block 110 to the femur 10.

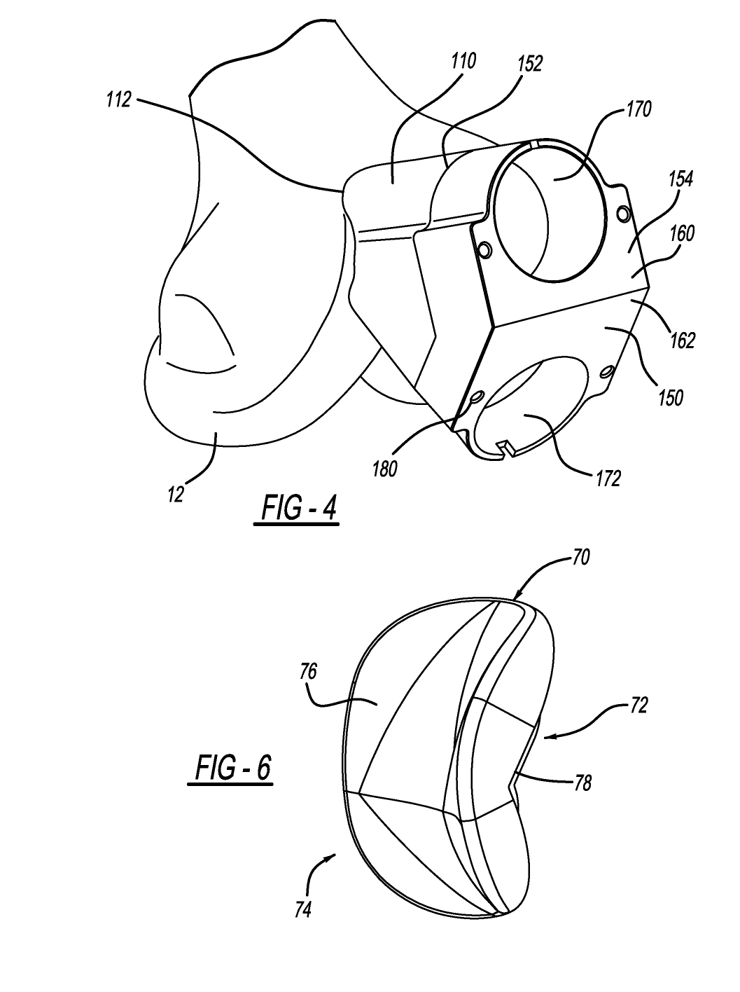

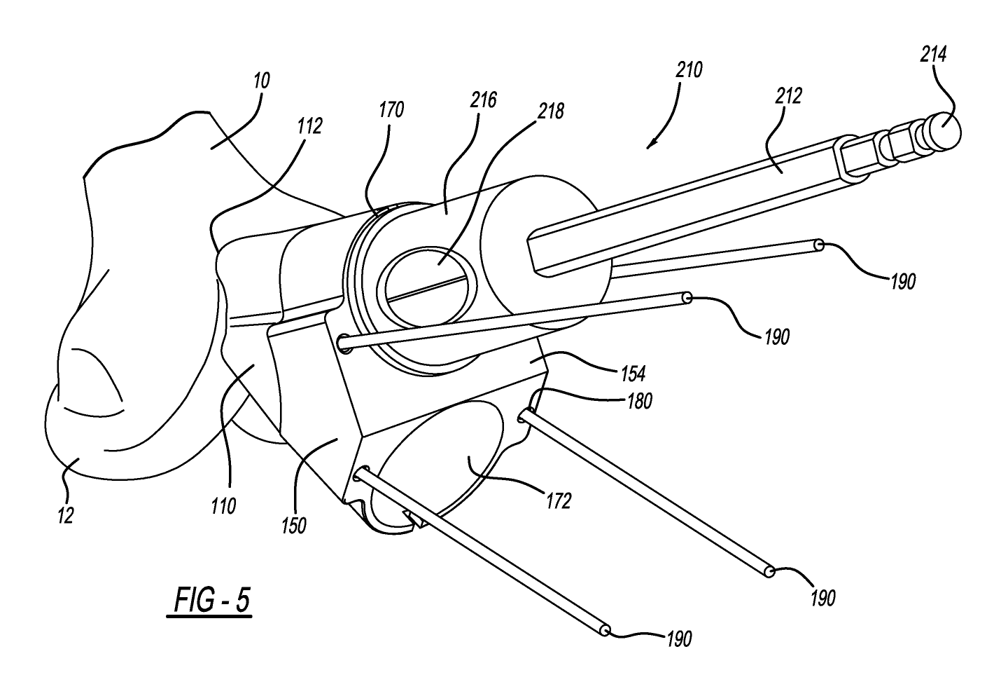

With reference to FIGS. 4 and 5, the present teachings further provide for a drill guide 150. The drill guide 150 generally includes a first or riser-engaging side 152, and a second or outer side 154. The riser-engaging side 152 is opposite to the outer side 154. The riser-engaging side 152 is sized and shaped to mount with the drill guide-engaging side 114 of the riser block 110. Both the drill guide-engaging side 114 and the riser-engaging side 152 are patient-generic. Thus, the riser-engaging side 152 of the drill guide 150 is configured to mate with the drill guide-engaging side 114 of a plurality of riser blocks 110, regardless of the configuration of the bone-engaging side 112 thereof. The drill guide 150 may thus be reused for multiple surgical procedures.

The outer side 154 of the drill guide 150 further includes a superior surface 160 and an inferior surface 162. The superior and inferior surfaces 160 and 162 extend away from one another at an angle towards the riser-engaging side 152. The riser-engaging side 152 is also angled, so as to match the angle of the superior and inferior surfaces 120 and 122.

The drill guide 150 further includes, and defines, a superior drill guide hole 170 and an inferior drill guide hole 172. Both the superior and inferior drill guide holes 170 and 172 extend through the drill guide 150 between the riser-engaging side 152 and the outer side 154. The superior drill guide hole 170 is positioned such that it is aligned with the superior riser block guide hole 130 when the drill guide is coupled to the riser block 110 as described herein. Similarly, the inferior drill guide hole 172 is arranged such that it is aligned with the inferior riser block guide hole 132 when the drill guide 150 is coupled to the riser block 110. The superior and inferior drill guide holes 170 and 172 each provide a bearing surface to interfere with a bushing of a reamer or drill, such as to limit the depth of the reamer or drill. The superior and inferior riser block guide holes 130 and 132 can be larger than the superior and inferior drill guide holes 170 and 172 respectively, such as to provide clearance for the reamer or drill.

The drill guide 150 further includes, and defines, guide bores 180. The guide bores 180 extend through the drill guide 150 between the riser-engaging side 152 and the outer side 154. The guide bores 180 of the drill guide 150 are positioned to align with the guide bores 140 of the riser block 110 when the riser-engaging side 152 of the drill guide 150 is mated with the drill guide-engaging side 114 of the riser block 110. Suitable coupling members, such as guide pins 190, can be inserted through the guide bores 180 and the guide bores 140 in order to couple the drill guide 150 and the riser block 110 together, as illustrated in FIG. 5, for example. The drill guide 150 can have any suitable shape and size to mate with the riser block 110. A plurality of drill guides 150 can thus be provided having different sizes and shapes. A drill guide 150 can be selected from a plurality of the drill guides 150 based on size and shape to mate with the riser block 110 manufactured for a particular patient.

With reference to FIG. 5, the riser block 110 is mounted to the femur 10 over the cartilage defect 20. The bone-engaging side 112, as a negative of the patient's femur 10, is configured to mate and nest with the femur 10 (and/or with cartilage thereof) at only a single location specific to the patient. In the patient-specific mounting location, the riser block 110 will be located such that when the drill guide 150 is coupled thereto, the superior and inferior drill guide holes 170 and 172, as well as the superior and inferior riser block guide holes 130 and 132, will direct a suitable cutting device, such as reamer 210, to the femur 10 in order to prepare the femur to receive the femoral implant 70 at the best fit location determined based on matching the implant model 60 with the femur image 50.

The riser block 110 is coupled to the femur 10 with any suitable coupling member, such as with guide pins 190 extending through the guide bores 140 of the riser block 110. The drill guide 150 is mated with the riser block 110, but with the guide pins 190 extending through the guide bores 180 of the drill guide 150.

The riser block 110 can be made of any suitable material, such as any suitable polymer. The drill guide 150 can also be made of any suitable material, such as steel, or any other material that is sterilizable. The drill guide 150 is typically more durable than the riser block 110, such as because the drill guide 150 cooperates with the reamer 210. Due to the drill guide's durability 150, it can be reused for multiple procedures. The riser block 110, being made of a polymeric material, can often be made more cost-effectively, which is particularly advantageous because the riser block can only be used with a single patient due to the patient-specific bone-engaging side 112.

The reamer 210 can be any suitable reamer for being guided to the femur 10 by the drill guide 150 and the riser block 110. The reamer 210 generally includes a shaft 212 with a driving end 214 at a proximal end thereof, and a suitable cutting member at a distal end thereof, which is opposite to the driving end 214 and is obstructed by the drill guide 150 and the riser block 110 of FIG. 5. To control a depth of the reamer 210, the reamer 210 can include a stop collar 216. The stop collar 216 is configured to mate with the superior drill guide hole 170 and the inferior drill guide hole 172 at the outer side 154 in order to limit the distance that the reamer 210 is inserted through the superior drill guide hole and the inferior drill guide hole 170 and 172, which limits the depth that the reamer 210 can be driven into the femur 10. The reamer 210 further includes a locking feature 218. The locking feature 218 can be, for example, a push button configured to provide for axial adjustment and selective locking of the stop collar 216 axially along the shaft 212 in order to set the maximum depth to which the reamer 210 is able to ream into the femur 10. After the femur 10 is reamed through the superior and inferior drill guide holes 170 and 172, such as to provide two adjoining holes in the femur 10, the implant site is finished in any suitable manner, such as with a chisel to further adjoin bone holes drilled through the superior and inferior drill guide holes 170 and 172 and smooth the outer edges of the implant site.

After the femur 10 has been prepared to receive the femoral implant 70, the femoral implant 70 is seated at the prepared site of the femur 10 and secured thereto in any suitable manner, such as with bone cement configured to secure the projection 78 of the femoral implant 70 to the femur 10. The patient-specific bone-engaging side 112 of the riser block 110 advantageously guides the reamer 210 to prepare the femur 10 such that the selected femoral implant 70 is implanted with the articular surface 76 thereof generally flush with the surrounding cartilage in order to provide improved articulation between the femur 10 and the patient's tibia.

A method of preparing the femur 10 to receive the femoral implant 70 to repair the cartilage defect 20 will now be described. A particular patient's femur 10 including cartilage defect 20 is first imaged in any suitable manner, such as by MRI imaging, x-ray imaging, or CT imaging. Femur image 50 illustrated in FIG. 2 is generated based on the imaged femur 10 of the particular patient in any suitable manner, such as described above. Based on the femur image 50, various shapes and sizes of the femoral implant 70 can be selected and/or trialed. For example, the implant model 60 can be positioned on the femur 10 in the area of the cartilage defect 20 in order to determine if the implant model 60 provides a best fit for the patient. Specifically, the implant model 60 will be arranged to determine if the articular surface 76 thereof is generally coplanar with cartilage surrounding the cartilage defect 20. A plurality of the implant models 60 can be moved to the femur 10 and the cartilage defect 20 to determine which one of the imaged implants provides a best fit. Each one of the imaged implants has a different size and/or shape, representing different sizes and/or shapes of femoral implants 70. The determination as to which one of the implant models 60, and corresponding femoral implants 70, provide the best fit at the femur 10 and cartilage defect 20 can be made by the patient's physician, the operating surgeon, or any other suitably trained personnel. For example, the determination may be made by a manufacturer and/or supplier of the femoral implant 70, the riser block 110, and/or the drill guide 150.

The riser block 110 is then prepared such that the bone-engaging side 112 thereof, which is patient-specific, is sized and shaped to mate with the patient's femur 10 at only a single position, the single position being an optimal position for guiding a suitable cutting instrument, such as the reamer 210, to the femur 10 in order to prepare the femur 10 to receive the selected femoral implant 70. The riser block 110 can be made in any suitable manner, using any suitable material. For example, the riser block 110 can be made using any suitable additive manufacturing process, such as 3D printing. Because the bone-engaging side 112 is patient-specific, the riser block 110 is suitable for one-time use, and is disposable.

During the surgical procedure to implant the femoral implant 70, the riser block 110 is seated on the patient's femur 10 such that the bone-engaging side 112 contacts the patient's femur 10, the bone-engaging side 112 being the patient-specific side so as to arrange the riser block 110 in position to guide the reamer 210 to the femur 10 to receive the femoral implant 70. The drill guide 150 is mated with the riser block 110 such that the riser-engaging side 152 of the drill guide 150 abuts and mates with the drill engaging side 114 of the riser block 110. The riser block 110 and the drill guide 150 can be coupled to the femur 10 in any suitable manner, such as with the guide pins 190, or any other suitable coupling members.

The bone is then prepared using any suitable cutting device, such as the reamer 210. Specifically, the reamer 210 is used to prepare a superior portion of the femur 10 by inserting the reamer shaft 212, which includes a suitable cutting member at a distal end thereof (obstructed in the figures) by the riser block 110 and the drill guide 150, through the superior drill guide hole 170 and the superior riser block guide hole 130 of the drill guide 150 and the riser block 110, respectively. The shaft 212 is driven at driving end 214 using any suitable driving device. The reamer 210 also prepares an inferior portion of the femur 10 by inserting the reamer through the inferior drill guide hole 172 and the inferior riser block guide hole 130 of the drill guide 150 and the riser block 110 respectively. The riser block 110 can have a patient-specific height in order to control the depth that the reamer 210 reams the femur 12. After the femur has been prepared, the guide pins 190, the drill guide 150, and the riser block 110 are removed from the femur 10, and the femoral implant 70 is implanted in the femur 10 at the prepared location.

The femoral implant 70 can be implanted in any suitable manner using any suitable fixation technique. For example, the femoral implant 70 can be secured within the femur 10 using any suitable bone cement. The femoral implant 70 is arranged in the femur 10 such that the projection 78 at the bone-engaging side 72 extends into the femur at the prepared surface thereof. The femoral implant 70 is arranged in the femur 10 such that the articular surface 76 thereof is generally flush or coplanar with the cartilage surrounding the cartilage defect 20.

Because the drill guide 150 is patient-generic, it can be used with a plurality of additional medical procedures. For example, the riser-engaging side 152 of the drill guide 150 can mate with the drill guide-engaging side 114 of any riser block 110 even though the bone-engaging side 112 of the riser block 110 is patient-specific. Thus, while a new and custom patient-specific riser block 110 will likely be required for additional procedures, the drill guide 150 can be sterilized and reused as long as the riser-engaging side 152 thereof and the drill guide-engaging side 114 of the riser block 110 are sized and shaped to mate with one another.

The present teachings thus provide numerous advantages. For example, the patient-specific surface of the bone-engaging side 112 of the riser block 110 ensures that the riser block 110 will be properly positioned to guide the reamer 210 to receive the implant 70 in a proper position such that the articular surface 76 thereof is generally flush or coplanar with surrounding cartilage. Furthermore, the present teachings reduce the need to prepare multiple femoral implants 70 of different sizes and shapes, and providing multiple femoral implants 70 in a kit, for example, in order to ensure that a femoral implant 70 providing a best fit is available. Instead, the femoral implant 70 can be manufactured “on demand” as necessary for a particular patient. Furthermore, the drill guide 150 can be reused as explained above, because the drill guide 150 can mate with the drill guide-engaging side 114 of multiple riser blocks 110 regardless of the configuration of the bone-engaging side 112 thereof.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for repairing a cartilage defect comprising:
a riser block including: a bone-engaging side that is a patient-specific negative of a patient's bone anatomy proximate to the cartilage defect; a drill guide-engaging side that is opposite to the bone-engaging side; a superior riser block guide hole defined by the riser block and extending from the drill guide-engaging side to the bone-engaging side; and an inferior riser block guide hole defined by the riser block and extending from the drill guide-engaging side to the bone-engaging side.

2. The system of claim 1, wherein the bone-engaging side is based on an image of a specific patient's bone.

3. The system of claim 1, wherein the drill guide-engaging side includes a superior surface and an inferior surface adjacent to the superior surface, the superior and inferior surfaces are sloped away from one another.

4. The system of claim 3, wherein the superior surface defines an opening of the superior guide hole, and the inferior surface defines an opening of the inferior guide hole.

5. The system of claim 1, wherein the riser block defines a plurality of guide bores extending therethrough configured to receive guide pins for securing the riser block to bone.

6. The system of claim 1, further comprising a drill guide.

7. The system of claim 6, wherein the drill guide is patient generic.

8. The system of claim 7, wherein the drill guide includes a riser-engaging side and an outer side that is opposite to the riser-engaging side, the riser-engaging side is shaped complementary to the drill guide-engaging side of the riser block to allow the drill guide to mate with the riser block.

9. The system of claim 8, wherein the drill guide defines a superior drill guide hole and an inferior drill guide hole, each of which extends from the outer side to the riser-engaging side of the drill guide; and
wherein when the riser-engaging side of the drill guide is mated with the drill guide-engaging side of the riser block, the superior drill guide hole is aligned with the superior riser block guide hole and the inferior drill guide hole is aligned with the interior riser block guide hole.

10. The system of claim 9, wherein the riser-engaging side of the drill guide is configured to mate with a plurality of different riser blocks with differently configured bone-engaging sides; and
wherein the drill guide is configured for use with a plurality of different surgeries performed on different patients.

11. The system of claim 10, further comprising a reamer configured to: be received within the inferior drill guide hole and the inferior riser block guide hole to direct the reamer to ream an inferior portion of the bone; be received within the superior drill guide hole and the superior riser block guide hole to direct the reamer to ream a superior portion of the bone; and cooperate with a bearing surface of the drill guide to limit a depth of the reamer;
wherein the inferior riser block guide hole and the superior riser block guide hole each have a diameter that is larger than a diameter of the inferior drill guide hole and the superior drill guide hole respectively.

12. A system for repairing a cartilage defect comprising:
a riser block including: a patient-specific bone-engaging side; a drill guide-engaging side that is opposite to the patient-specific bone-engaging side; a superior riser block guide hole; and an inferior riser block guide hole, both the superior and inferior riser block guide holes are defined by and extend through the riser block from the drill guide-engaging side to the patient-specific bone-engaging side;
a drill guide including: an outer side; a riser-engaging side opposite to the outer side, the riser-engaging side is complementary to the drill guide-engaging side of the riser block; a superior drill guide hole; and an inferior drill guide hole, both of the superior and inferior drill guide holes extend through the riser block and are arranged to align with the superior and inferior riser block guide holes respectively when the drill guide is coupled to the riser block; and
a reamer configured to be guided to the bone surface by the superior and inferior drill guide holes, and the superior and inferior riser block guide holes, to prepare the bone to receive an implant.

13. The system of claim 12, wherein the patient-specific bone-engaging side is based on an image of a specific patient's bone.

14. The system of claim 12, wherein the superior drill guide hole and the superior riser block guide hole define a superior guide passageway, and the inferior drill guide hole and the inferior riser block guide hole define an inferior guide passageway; and
wherein the superior guide passageway and the inferior guide passageway are angled relative to one another such that the superior guide passageway and the inferior guide passageway are closer together in the riser block than in the drill guide.

15. The system of claim 12, wherein the riser-engaging side of the drill guide is configured to mate with a plurality of different riser blocks with differently configured patient-specific bone-engaging sides; and
wherein the drill guide is configured for use with a plurality of different surgeries performed on different patients.

16. The system of claim 12, further comprising a plurality of coupling members configured to extend through guide bores defined by both the riser block and the drill guide, and extend into a bone surface proximate to the cartilage defect to secure the riser block and the drill guide to the bone with the riser block between the drill guide and the bone.

17. A method for repairing a cartilage defect, the method comprising:
imaging a bone of a patient proximate to the cartilage defect;
positioning a riser block on the bone proximate to the cartilage defect such that a patient-specific bone-engaging side of the riser block formed based on the imaging mates with the bone;
positioning a drill guide such that a riser-engaging side of the drill guide abuts a drill guide-engaging side of the riser block, and such that superior and inferior drill guide holes defined by the drill guide are respectively aligned with superior and inferior riser block guide holes of the riser block;
coupling the riser block and the drill guide together and to the bone with coupling members extending through the drill guide, through the riser block, and into the bone;
inserting a reamer through the superior drill guide hole and the superior riser block guide hole to guide the reamer to a superior portion of the bone, and reaming the superior portion of the bone to receive an implant;
inserting the reamer through the inferior drill guide hole and the inferior riser block guide hole to guide the reamer to a superior portion of the bone, and reaming the superior portion of the bone to receive the implant; and
implanting the implant in the bone such that an articular surface of the implant is generally flush with surrounding cartilage.

18. The method of claim 17, further comprising forming the riser block.

19. The method of claim 17, further comprising reusing the drill guide during an additional surgical procedure.

20. The method of claim 17, further comprising virtually trialing the implant with a computer.

21. The method of claim 17, further comprising forming the riser block such that the patient-specific bone-engaging side is a negative of the patient's bone surface.

22. The method of claim 17, further comprising selecting the drill guide from a plurality of different drill guides of different sizes such that the selected drill guide is configured to mate with the riser block.
